(19) United States
(12) Patent Application Publication
(10) Pub. No.: US 2024/0002934 A1
(43) Pub. Date: Jan. 4, 2024

(54) COMPOSITION FOR DETECTING TARGET NUCLEIC ACID AND METHOD FOR DETECTING TARGET NUCLEIC ACID USING SAME

(71) Applicant: KOREA RESEARCH INSTITUTE OF BIOSCIENCE AND BIOTECHNOLOGY, Daejeon (KR)

(72) Inventors: Eun Kyung LIM, Daejeon (KR); Jae Woo LIM, Daejeon (KR); Byung Hoon KANG, Daejeon (KR); Tae Joon KANG, Daejeon (KR); Seung Beom SEO, Daejeon (KR); Soo Jin JANG, Daejeon (KR); Ju Yeon JUNG, Daejeon (KR)

(73) Assignee: KOREA RESEARCH INSTITUTE OF BIOSCIENCE AND BIOTECHNOLOGY, Daejeon (KR)

(21) Appl. No.: 18/037,222

(22) PCT Filed: Nov. 3, 2021
(86) PCT No.: PCT/KR2021/015826

§ 371 Date: May 16, 2023

(30) Foreign Application Priority Data
Nov. 16, 2020 (KR) 10-2020-0153050

(51) Int. Cl. C12Q 1/6876; C12Q 1/6806

(52) U.S. Cl. CPC C12Q 1/6876; C12Q 1/6806

(57) ABSTRACT

The present invention relates to a technology for analyzing and detecting/diagnosing a target nucleic acid. When the detection system according to the present invention is used, effective real-time detection or diagnosis efficiency can be obtained while problems such as noise are minimized. In particular, since, by using a house-keeping gene according to a method of use, the expression difference of the target nucleic acid can be corrected, and direct real-time target nucleic acid detection is possible, it can be effectively used for detecting various nucleic acids and diagnosing various diseases thereby.

### TECHNICAL FIELD

The present invention relates to a technology for analyzing and detecting/diagnosing a target nucleic acid.

### BACKGROUND ART

Molecular diagnosis is a diagnostic method that detects or analyzes nucleic acids such as DNA or RNA, and has the advantage of being very accurate and obtaining a lot of information compared to other diagnostic methods since the specificity of base sequences is used. In addition, molecular diagnosis is a field with a large market size and a fast growth rate due to a very wide range of applications, such as cancer diagnosis, diagnosis of human or livestock infectious diseases, pathogen antibiotic resistance test, food test, blood test, genetic test, and the like. In particular, on-site molecular diagnosis is the most active research field because the molecular diagnosis is able to expand the area of molecular diagnosis through strengthening access to medical support, immediate analysis and prescription of results, reduction in the number of hospital visits and waiting time, and the like.

In particular, a method of labeling and detecting nucleic acids that are difficult to be detected in natural state thereof has been applied to various fields of molecular biology or cell biology. Nucleic acids with labeled substances attached have been widely used in order to detect signals on southern blotting, northern blotting, in situ hybridization, and nucleic acid microarrays using specific hybridization reactions. A method of amplifying DNA and simultaneously labeling DNA using labeled monomers (labeled dNTPs) or labeled primers in a polymerase chain reaction (PCR) is known. The thus labeled DNA is able to be detected with a microarray.

The method of labeling nucleic acids while simultaneously performing PCR has an advantage of not requiring a separate step for labeling, but has a disadvantage in that when a monomer labeled with a fluorescent dye or the like is used, PCR efficiency is lower than using an unlabeled monomer. In addition, since RNA is not able to be amplified by PCR, detecting RNA by PCR labeling requires a step of preparing cDNA through reverse transcription, and in particular, short RNAs such as microRNAs (miRNAs) have a problem in that cDNA preparation is cumbersome. Accordingly, there is an urgent need to develop a nucleic acid detection technology having more improved sensitivity and specificity.

The methods described above are easy to detect a nucleic acid to be targeted when a large amount of detection nucleic acid is present, and have been widely used today. Nevertheless, when a small amount of target nucleic acid is present, it is very difficult to detect the nucleic acid (low sensitivity), and there are frequent cases that due to other inhibitors, it is impossible to detect a specific target only, but a non-specific target is incorrectly detected (low specificity).

On the other hand, catalytic hairpin assembly, which is an isothermal and non-enzyme-free signal amplification reaction is a reaction that produces a large amount of double-stranded products in the form of a combination of two types of hairpin probes by acting a single-stranded nucleic acid as a catalyst to repeatedly perform strand displacement reactions on two types of metastable hairpin probes, which has been used in the development of detection technologies for various biomaterials.

However, there has been no development of a specific system for high-sensitivity detection using the catalytic hairpin assembly or the system for commercial use.

### DISCLOSURE

### Technical Problem

The present inventors have made diligent efforts to develop a rapid and accurate detection method, and as a result, have developed a detection system comprising two types of probes spaced apart in different liposomes, and completed the present invention. More specifically, the present inventors manufactured a system in which when liposomes comprising individual probes supported thereon are degraded by a buffer solution (reaction buffer) containing a surfactant, the probes are released from each liposome to perform a mutual reaction. As the two types of probes participating in the reaction are spaced apart as described above, it is possible to exhibit effective real-time diagnosis efficiency while minimizing problems such as noise caused by interference between probes.

Therefore, the present invention provides a composition for detecting a target nucleic acid comprising: 1) a first probe having a hairpin structure in which a reporter is conjugated to one end and a quencher is conjugated to the other end, and comprising an off-target sequence and a target sequence complementary to the target nucleic acid; and 2) a second probe comprising a sequence complementary to the first probe, wherein the first probe and the second probe are each supported on separate liposomes, a kit using the same, and a method for detecting a target nucleic acid.

### Technical Solution

In one general aspect, the present invention provides a composition for detecting a target nucleic acid comprising: 1) a first probe having a hairpin structure in which a reporter is conjugated to one end and a quencher is conjugated to the other end, and comprising an off-target sequence and a target sequence complementary to the target nucleic acid; and 2) a second probe comprising a sequence complementary to the first probe; wherein the first probe and the second probe are each supported on separate liposomes.

Further, the present invention provides a hydrogel for detecting a target nucleic acid comprising: 1) a first probe having a hairpin structure in which a reporter is conjugated to one end and a quencher is conjugated to the other end, and comprising an off-target sequence and a target sequence complementary to the target nucleic acid; and 2) a second probe comprising a sequence complementary to the first probe, wherein the first probe and the second probe are each supported on separate liposomes.

In addition, the present invention provides a kit for detecting a target nucleic acid comprising the composition or hydrogel for detection as described above.

Further, the present invention provides a method for detecting a target nucleic acid, comprising: reacting a sample and a surfactant with 1) a first probe having a hairpin structure in which a reporter is conjugated to one end and a quencher is conjugated to the other end, and comprising an off-target sequence and a target sequence complementary to the target nucleic acid; and 2) a second probe comprising a sequence complementary to the first probe; wherein the first probe and the second probe are each supported on liposomes that are degraded by the surfactant.

In addition, the present invention provides a sensor for detecting a target nucleic acid comprising: an inlet part; at least one sensing part containing the composition for detecting a target nucleic acid as described above; and a passage part connecting the inlet part and the sensing part.

### Advantageous Effects

When the detection system according to the present invention is used, effective real-time detection or diagnosis efficiency may be obtained while problems such as noise are minimized. In particular, since, by using a house-keeping gene according to a method of use, the expression difference of the target nucleic acid may be corrected, and direct real-time target nucleic acid detection is possible, it can be effectively used for detecting various nucleic acids and diagnosing various diseases thereby.

### DESCRIPTION OF DRAWINGS

FIG. 1 shows (a) a schematic diagram of the catalytic hairpin assembly (CHA) reaction and (b) results of reacting designed probes according to the present invention.

FIG. 2 shows fluorescence results of confirming the reaction by the target sequence (Target) and the probes (A, B) used in the CHA reaction. FIG. 2a shows results of the change in fluorescence expression according to reactions between the probes and the target sequence. FIG. 2b shows results of the change in fluorescence expression according to the reaction between the probes and the target sequence and mutant target sequences (1MS, 2MS). FIGS. 2c and 2d show results of the detection limit while changing the concentration of the target sequence from 10 nM to 100 fM.

FIG. 3 shows that the probe was encapsulated in the liposome.

FIG. 4 shows a mold manufacturing process for producing a hydrogel according to the present invention (a) and hydrogel production results (b and c).

FIG. 5 shows a schematic diagram of a microfluidic chip according to the present invention.

FIG. 6 shows a schematic diagram of a reaction using the microfluidic chip according to the present invention.

FIG. 7 shows the configuration for the entire reaction of the microfluidic chip according to the present invention and the specific configuration of a sensing part therein.

FIG. 8 shows that the probes encapsulated in the liposomes according to the present invention are released by treatment with a surfactant and participate in a reaction. FIG. 8a shows a schematic diagram of the reaction process, FIG. 8b shows a change in fluorescence expression according to the reaction, FIG. 8c shows a micrograph of liposomes taken before the surfactant treatment, and FIG. 8(d) shows a micrograph of liposomes taken after the surfactant treatment. FIGS. 8e and 8f show changes in fluorescence response dependent on the concentration of the target nucleic acid.

FIG. 9 shows results of the diffusion change of the probe depending on the change in hydrogel composition (by changing the PEG conditions) confirmed by using FITC (a) and Cy5 (b)

FIG. 10 shows results of a change in the response level depending on the change in the hydrogel composition (by changing the PEG conditions).

FIG. 11 shows expression changes of HRBB2 in cells and exosomes according to the cell lines.

FIG. 12 shows confirmation results of the expression change of ERBB2 together with the house-keeping gene in the cell lines HCC1954 and HCC1143.

FIG. 13 shows the tumor size by cycle in nude mice prepared using the cell line HCC1954.

FIG. 14 shows the expression change of ERBB2 using exosomes isolated from mouse urine. FIG. 14a shows quantitative results of the ERBB2 gene expression in mouse urine-derived exosomes. FIG. 14b shows the fluorescence measurement result measured after treating mouse urine-derived exosomes on the hydrogel of the present invention, FIG. 14c shows quantification results thereof, and FIG. 14d shows results corrected by the GAPDH fluorescence value.

FIG. 15 shows results of sequence specificity for mismatch sequences and the target sequence using a hydrogel-based gene detection composition.

FIG. 16 shows the expression change of ERBB2 in exosomes isolated from mice using the microfluidic chip according to the present invention.

FIG. 17 shows detection progress results for the presence or absence of ERBB2 and GAPDH using the microfluidic chip according to the present invention.

FIG. 18 shows detecting results of ERBB2 expressed in exosomes derived from cell lines HCC1143 and HCC1954 using the microfluidic chip according to the present invention.

FIG. 19 shows the expression change of ERBB2 expressed in exosomes derived from mouse blood using the microfluidic chip according to the present invention.

FIG. 20 shows a schematic diagram of components of a microfluidic chip according to the present invention.

### BEST MODE

The present invention provides a composition for detecting a target nucleic acid, and a method and a kit for detecting a target nucleic acid using the same, characterized in that first and second probes capable of detecting the target nucleic acid are supported on separate liposomes. By using the detection system of the present invention, it is possible to minimize noise generated by mutual interference between the probes and perform accurate real-time diagnosis and detection.

Hereinafter, the present invention will be described in more detail.

The present invention provides a composition for detecting a target nucleic acid comprising: 1) a first probe having a hairpin structure in which a reporter is conjugated to one end and a quencher is conjugated to the other end, and comprising an off-target sequence and a target sequence complementary to the target nucleic acid; and 2) a second probe comprising a sequence complementary to the first probe, wherein the first probe and the second probe are each supported on separate liposomes.

In the present invention, the first probe may be used as a detection probe by comprising a target sequence complementary to the target nucleic acid and specifically binding to the target nucleic acid in the presence thereof to exhibit a fluorescence reaction.

The off-target sequence included in the first probe is not a detection sequence that complementarily hybridizes to the target gene, but refers to a sequence capable of complementarily binding with the target sequence to form a hairpin structure, and also capable of complementarily binding with some sequences of the second probe.

More specifically, the first probe has a reporter conjugated to one end and a quencher conjugated to the other end in order to show a change in fluorescence expression depending on the presence or absence of the target nucleic acid, but is not limited thereto. In a preferred embodiment of the present invention, a first probe in which a reporter is conjugated to the 5′ end and a quencher is conjugated to the 3′ end was used.

In the present invention, the reporter may independently have a fluorescent group. For example, the reporter may have a fluorescent group such as ALEX-350, FAM, VIC, TET, CAL Fluor® Gold 540, JOE, HEX, CAL Fluor Orange 560, TAMRA, CAL Fluor Red 590, ROX, CAL Fluor Red 610, TEXAS RED, CAL Fluor Red 635, Quasar 670, CY3, CY5, CY5.5, or Quasar 705.

In addition, as used herein, the quencher is a molecule or a group capable of absorbing/quenching fluorescence. For example, groups such as DABCYL, BHQ (for example, BHQ-1 or BHQ-2), ECLIPSE, and/or TAMRA may be used.

In the present invention, the second probe is characterized by comprising a sequence complementary to the first probe and may have a hairpin structure.

The hairpin structure may be naturally occurring or may be artificially introduced. For example, the detection probe may have a hairpin structure formed by adding two complementary oligonucleotide sequences to the two ends of the detection probe. In such embodiments, the two complementary oligonucleotide sequences form an arm (stem) having a hairpin structure. The arm having a hairpin structure may have any desired length, for example, 2-15 nt, for example, 3-7 nt, 4-9 nt, 5-10 nt, or 6-12 nt.

More specifically, the second probe may comprise the target sequence and a sequence complementary to the off-target sequence of the first probe, and the first probe and the second probe may be used for catalytic hairpin assembly (CHA). When the detection reaction of the present invention is initiated, the target nucleic acid starts fluorescence recovery and catalyzes the assembly of the first probe and the second probe through a toehold-mediated hairpin DNA circuit. The target nucleic acid repeatedly causes a target nucleic acid strand displacement reaction in the first and second probes, which are two types of meta-stable hairpin probes, thereby making possible to generate a large amount of a double-stranded product in which the two types of hairpin probes are bound.

In the present invention, ‘target nucleic acid’ refers to all kinds of nucleic acids to be detected, and may or may not include a mutant gene. It is characterized by comprising all kinds of DNA, comprising genomic DNA, mitochondrial DNA, and viral DNA, or all kinds of RNA, comprising mRNA, ribosomal RNA, non-cording RNA, tRNA, and viral RNA, but is not limited thereto. The target nucleic acid is annealed to or hybridized with the primer or probe under hybridization, annealing or amplification conditions.

As used herein, the liposome comprising the first and second probes is a spherical vesicle structure formed of one or more artificially made lipid bilayers.

The lipid forming the liposome of the present invention is not particularly limited and may be a known lipid. The lipid may comprise, for example, phospholipids, glycolipids, sterols, cationic lipids, and the like, polyglycerol alkyl ethers, polyoxyethylene alkyl ethers, alkyl glycosides, alkylmethyl glucamides, alkyl sucrose esters, dialkyl polyoxyethylene ether, dialkyl polyglycerol ether, and the like, amphiphilic block copolymers such as polyoxyethylene-polylactic acid, and the like, long-chain alkylamines or long-chain fatty acid hydrazides, and the like.

For example, the lipid is preferably at least one of natural or synthetic phospholipids such as phosphatidylcholine (such as soybean phosphatidylcholine, egg yolk phosphatidylcholine, bovine phosphatidylcholine, dilauroylphosphatidylcholine, dimyristoylphosphatidylcholine, dipalmitoylphosphatidylcholine or distearoylphosphatidylcholine, or the like), phosphatidylethanolamine (such as dilauroylphosphatidylethanolamine, dimyristoylphosphatidylethanolamine, dipalmitoylphosphatidylethanolamine or distearoylphosphatidylethanolamine, dioleoylphosphatidylethanolamine, or the like), phosphatidylserine (such as dilauroylphosphatidylserine, dimyristoylphosphatidylserine, dipalmitoylphosphatidylserine or distearoylphosphatidylserine, or the like), phosphatidic acid, phosphatidylglycerol (such as dilauroylphosphatidylglycerol, dimyristoylphosphatidylglycerol, dipalmitoylphosphatidylglycerol or distearoylphosphatidylglycerol, or the like), phosphatidylinositol (such as dilauroylphosphatidylinositol, dimyristoylphosphatidylinositol, dipalmitoylphosphatidylinositol or distearoylphosphatidylinositol, or the like), lysophosphatidylcholine, sphingomyelin, egg yolk lecithin, soy lecithin, hydrogenated phospholipids, and the like.

Examples of the glycolipid may comprise glyceroglycolipids, sphingoglycolipids, and the like. Examples of the glyceroglycolipid may comprise digalactosyl diglycerides (digalactosyl dilauroyl glyceride, digalactosyl dimyristoyl glyceride, digalactosyl dipalmitoyl glyceride or digalactosyl distearoyl glyceride, and the like) or galactosyl diglycerides (galactosyl dilauroyl glyceride, galactosyl dimyristoyl glyceride, galactosyl dipalmitoyl glyceride or galactosyl distearoyl glyceride, and the like), and the like. Examples of the sphingoglycolipid may comprise galactosyl cerebroside, lactosyl cerebroside, ganglioside, and the like.

The sterols may be cholesterol, cholesterol hexasuccinate, 3β-[N—(N′,N′-dimethylaminoethane)carbamoyl]cholesterol, ergosterol, or lanosterol.

The cationic lipid may comprise dioctadecylamidoglycylspermidine (DOGS), dimethyldioctadecylammonium bromide (DDAB), L-a-dioleoyl phostatidylethanolamine (DOPE), [N—(N,N′-dimethylaminoethane)carbamoyl]cholesterol (DC-Chol), N-[1-(2,3-dioleyloxy)propyl]-N,N,N-trimethylammonium bromide (DOTMA), 2,3-dioleoyloxy-N-[2-(sperminecarboxamido-O-ethyl]-N,N-dimethyl-propanaminium trifluoroacetate (DOSPA), 1-[2-(oleoyloxy)-ethyl]-2-oleyl-3-(2-hydroxyethyl)imidazolinium chloride (DOTIM), 1,2-dimyristyloxypropyl-3-dimethyl-hydroxy ethyl ammonium bromide (DMRIE), 1,2-dimyristoyl-3-dimethylammonium propane (DMDAP), 1,2-dipalmitoyl-3-dimethylammonium propane (DPDAP), 1,2-dilauroyl-3-dimethylammonium propane (DLDAP), 1,2-distearoyl-3-dimethylammonium propane (DSDAP), 1,2-dioleoyl-3-dimethylammonium propane (DODAP), 1,2-dimyristyl-3-dimethylammonium propane (DMDAP), 1,2-dipalmityl-3-dimethylammonium propane (DPDAP), 1,2-dilauryl-3-dimethylammonium propane (DLDAP), 1,2-distearyl-3-dimethylammonium propane (DSDAP), 1,2-dioleyl-3-dimethylammonium propane (DODAP), 1,2-dimyristoyl-3-trimethylammonium propane (DMTAP), 1,2-dipalmitoyl-3-trimethylammonium propane (DPTAP), 1,2-dilauroyl-3-trimethylammonium propane (DLTAP), 1,2-distearoyl-3-trimethylammonium propane (DSTAP), dioleoyl-3-trimethylammonium propane (DOTAP), 1,2-dimyristyl-3-trimethylammonium propane (DMTAP), 1,2-dipalmityl-3-trimethylammonium propane (DPTAP), 1,2-dilauryl-3-trimethylammonium propane (DLTAP), 1,2-distearyl-3-trimethylammonium propane (DSTAP), 1,2-dioleyl-3-trimethylammonium propane (DOTAP), and the like.

The liposome-forming lipid may be used alone or in combination of two or more.

According to an embodiment of the present invention, the liposome may be prepared using a conventional production process. For example, a lipid membrane hydration method may be used. This method is to form a liposome by hydrating a lipid membrane, and any solution for hydrating the lipid membrane may be used without limitation as long as it is able to hydrate the lipid membrane.

According to an embodiment of the present invention, the liposome may be prepared by mixing phosphatidylcholine (PC), dioleoyl-3-trimethylammonium propane (DOTAP), and cholesterol (5-cholesten-3β-ol). It is preferable to mix and use phosphatidylcholine (PC), cholesterol (5-cholesten-3β-ol), and dioleoyl-3-trimethylammonium propane (DOTAP) in a molar ratio (PC:cholesterol:DOTAP) of about 1:0.2 to 0.8:0.05 to 0.2, preferably 1:0.5:0.1.

These liposomes may be used in any form as long as they are spherical vesicles formed of a lipid bilayer capable of supporting probes. Preferably, cationic liposomes may be used.

In the present invention, the composition for detecting a target nucleic acid may be a hydrogel composition. The hydrogel composition may be cured through a separate curing treatment, and in the cured hydrogel, liposomes comprising the first probe and the second probe supported thereon, respectively, may be spaced apart and included.

Each of the liposomes may be fixed to the porous structure of the hydrogel, and the multifaceted three-dimensional structure inside the hydrogel has an advantage in that the liposome is able to be immobilized inside the hydrogel without chemical binding. In addition, the porous structure is advantageous for foreign substances (genes or exosomes for diagnosis) to flow into the interior through diffusion, thereby facilitating contact between the sample and the liposome of the present invention.

Thus, the present invention provides a hydrogel for detecting a target nucleic acid comprising: 1) a first probe having a hairpin structure in which a reporter is conjugated to one end and a quencher is conjugated to the other end, and comprising an off-target sequence and a target sequence complementary to a target nucleic acid; and 2) a second probe comprising a sequence complementary to the first probe, wherein the first probe and the second probe are each supported on separate liposomes.

In the present invention, “hydrogel” is a concept encompassing a gel containing water as a basic component, a gel using water as a dispersion medium, or a hydrophilic gel.

Hydrogel particles may contain a hydrophilic monomer or polymer. In another aspect of the present invention, the hydrogel particles may contain at least one selected from the group consisting of natural polymer, acrylic monomer or polymer, polyacrylamide-based monomer or polymer, phosphatidyl choline, hyaluronic acid-based monomer or polymer, carboxymethyl cellulose, alginate, chitosan, poly(e-caprolactone), poly(lactic acid), poly(glycolic acid), polyethylene glycol, hydroxyapatite, tricalcium phosphate, and a mixture thereof.

The natural polymer comprises at least one selected from the group consisting of polysaccharides derived from red algae, such as carrageenan, agar, and agarose, polysaccharides containing mannose, such as mannan, galactomannan, glucomannan and derivatives thereof, and natural gums such as locust bean gum, guar gum, xanthan gum, gum arabic, gellan gum, and gum karaya.

The acrylic monomer or polymer comprises hydrophilic acrylic monomers or polymers, specifically at least one selected from the group consisting of polyethylene glycol diacrylate, polyethylene glycol methacrylate, polymethylmethacrylate (PMMA), hydroxyethyl acrylate (HEA), and hydroxyethyl methacrylate (HEMA).

In still another aspect of the present invention, the hydrogel particle may preferably contain an acrylic monomer or polymer capable of radical polymerization in order to secure wide usability, and specifically, may preferably contain a polyethylene glycol acrylate-based monomer or polymer.

More preferably, polyethylene glycol and a polyacrylamide-based monomer or polymer may be mixed and used. More specifically, at least one selected from the group consisting of polyethylene glycol; and polyethylene glycol diacrylate, polyethylene glycol methacrylate, polymethylmethacrylate (PMMA), hydroxyethyl acrylate (HEA), and hydroxyethyl methacrylate (HEMA) may be included, and more specifically, polyethylene glycol and polyethylene glycol diacrylate may be mixed and used. In other words, the hydrogel particle may contain a mixture of polyethylene glycol and polyethylene glycol diacrylate.

A mixing ratio of polyethylene glycol and polyethylene glycol diacrylate is preferably a weight ratio of 1:0.5 to 2, and more specifically, approximately 1:1. Even more preferably, polyethylene glycol and polyethylene glycol diacrylate may be mixed in a weight ratio of about 3:0.5 to 2:0.5 to 2 (hydrophilic aqueous solution:polyethylene glycol:polyethylene glycol diacrylate) in the hydrophilic aqueous solution, more preferably 3:1:1.

The polymer capable of forming the hydrogel is preferably a photocurable type, and more preferably photocurable by ultraviolet irradiation. In other words, the hydrogel may be produced by photocuring.

A photoinitiator may initiate free radical polymerization and/or crosslinking with the use of light. Examples of suitable photoinitiators comprise, but are not limited to, benzoin methyl ether, diethoxyacetophenone, benzoylphosphine oxide, 2-hydroxy-2-methyl propiophenone (HMPP), 1-hydroxycyclohexyl phenyl ketone, and Darocur (brand name) and Irgacure (brand name) types, preferably Darocur 1173 and 2959. Examples of benzoylphosphine initiators comprise 2,4,6-trimethylbenzoyl diphenylphosphine oxide; bis-(2,6-dichlorobenzoyl)-4-N-propylphenylphosphine oxide; and bis-(2,6-dichlorobenzoyl)-4-N-butylphenylphosphine oxide. For example, reactive photoinitiators capable of being incorporated into macromers, or capable of being used as specific monomers, are also suitable.

If a photoinitiator is contained, polymerization may be initiated by actinic radiation, for example by specific ultraviolet light having a suitable wavelength. Spectral requirements may be controlled, if appropriate, by the addition of suitable photosensitizers.

Further, the present invention provides a kit for detecting a target nucleic acid comprising the composition as described above, the composition comprising: 1) a first probe having a hairpin structure in which a reporter is conjugated to one end and a quencher is conjugated to the other end, and comprising an off-target sequence and a target sequence complementary to the target nucleic acid; and 2) a second probe comprising a sequence complementary to the first probe, wherein the first probe and the second probe are each supported on separate liposomes.

For the description of the composition of the kit, the description of the composition for detecting a target nucleic acid may be equally applied.

The kit of the present invention may comprise a composition for detecting a target nucleic acid in one compartment, and separately, may further comprise a surfactant in a separate compartment. The surfactant is provided together with the target nucleic acid when the detection reaction is initiated, and may react with the liposome included in the composition for detecting the target nucleic acid to degrade the liposome. The supported probes are released by degradation of the liposome due to the surfactant, and the released probes exhibit a fluorescence reaction depending on the presence or absence of the target nucleic acid, thereby making it possible to detect the target nucleic acid.

In other words, before being contacted with the surfactant, the probes of the present invention are spaced apart from each other and contained in individual liposomes, and only when the detection reaction is initiated, the probes are converted to a state in which mutual reactions are possible, thereby effectively removing noise that may occur before the reaction. In addition, through this, the reaction may be performed without an additional temperature change and temperature control device, the addition of enzymes or other substrates is unnecessary, and the reaction may proceed without requiring complicated and time-consuming experimental procedures.

Examples of surfactant capable of being used in the present invention may comprise, but are not limited to, cetyl trimethylammonium bromide, hexadecyl trimethyl ammonium bromide, dodecyl betaine, dodecyl dimethylamine oxide, 3-(N, N-dimethylpalmitylammonio) propane sulfonate), Tween 20, Tween 80, Triton-X-100, polyethylene glycol monooleyl ether, triethylene glycol monododecyl ether, octyl glucoside, N-nonanoyl-N-methylglucamine, and the like, as long as the purpose of liposome degradation is achieved.

According to an embodiment of the present invention, the surfactant may be Triton X-100.

The surfactant may be contained in a buffer solution in an amount of about 0.5% to 5% by weight, preferably 0.6% to 2% by weight, and more preferably about 1% by weight.

An optimal amount of reagents to be used in a particular reaction may be easily determined by those skilled in the art having the knowledge of the disclosure herein. Typically, the kit of the present invention is manufactured in a separate package or compartment comprising the above described components. In addition, the kit may further comprise instructions for use and other tools or equipment necessary for detection.

Further, the present invention provides a method for detecting a target nucleic acid, comprising: reacting a sample and a surfactant with 1) a first probe having a hairpin structure in which a reporter is conjugated to one end and a quencher is conjugated to the other end, and comprising an off-target sequence and a target sequence complementary to the target nucleic acid; and 2) a second probe comprising a sequence complementary to the first probe; wherein the first probe and the second probe are each supported on liposomes that are degraded by the surfactant.

The “sample” means any biological or environmental sample containing any DNA, RNA and/or target DNA, RNA. The biological sample may be any tissue or body fluid obtained from a subject. The biological sample comprises, but not limited to, a subject's sputum, blood, serum, plasma, blood cells (for example, white blood cells), tissues, biopsy samples, smear samples, rinse samples, swab samples, body fluids containing cells, mobile nucleic acid, urine, peritoneal fluid and pleural fluid, cerebrospinal fluid, feces, lacrimal fluid or cells therefrom. Biological samples may also comprise tissue sections taken for histological purposes, i.e., frozen or fixed sections or microdissected cellular or extracellular portions thereof. The biological sample may be obtained by a method that does not harm a subject.

Preferably, the sample may be mixed in a buffer solution containing a surfactant and provided together, and when the sample and the surfactant come into contact with the liposome containing the first probe or the second probe supported thereon, the surfactant may degrade the liposome, and thus the first probe and the second probe may be released to detect the presence or absence of the target nucleic acid in the sample.

In addition, in the present invention, the liposome containing the first probe and the liposome containing the second probe may be simultaneously contained in the hydrogel, and the first and second probes may be simultaneously released into the hydrogel by the surfactant and react with the sample on the hydrogel.

The method for detecting a target nucleic acid of the present invention may further comprise a step of visually confirming a change in fluorescence of a reactant; or measuring a change in fluorescence of a reactant. The measurement of the change in fluorescence may be a conventional fluorescence measurement method in which a measurement wavelength of a fluorescence device is fixed and measured. Specifically, the change in fluorescence may be confirmed by fixing the measurement wavelength of the fluorescence device. For example, in the case of FAM fluorescence, a method of measuring the fluorescence intensity of a reactant at a wavelength of ex; 495/em; 520 and observing fluorescence change at intervals of 5 to 10 minutes for 1 to 2 hours may be used.

Further, the present invention relates to a sensor for detecting a target nucleic acid comprising: an inlet part; at least one sensing part containing the composition for detecting a target nucleic acid as described above according to the present invention; and a passage part connecting the inlet part and the sensing part.

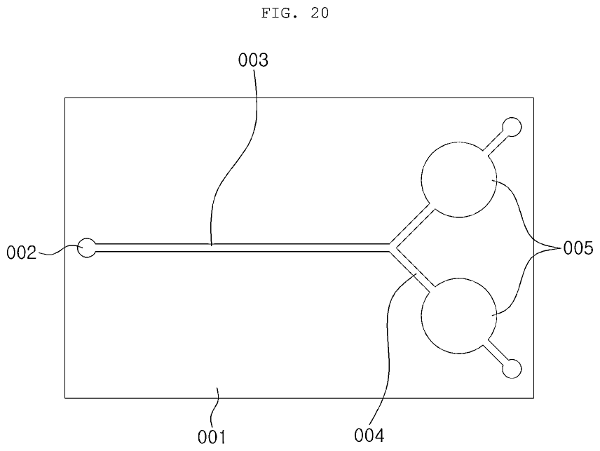

An exemplary structure of the sensor for detecting a target nucleic acid is shown in FIG. 20.

A sensor (001) for detecting a target nucleic acid is provided with an Inlet (002) located therein, and includes a passage (003) connecting the sample and/or the surfactant to move from the inlet to the outlet. The passage may be a microtubule passage. The sample and/or surfactant is injected from the inlet and moves toward the outlet through the passage, and a detection reaction occurs when the fluid comes into contact with the sensing part (005) located in the mobile phase.

The composition for detecting a target nucleic acid of the present invention may be included in the form of a hydrogel in the sensing part, and liposomes comprising individual probes supported thereon may be spaced apart and included in pores inside the hydrogel.

For the purpose of detecting a plurality of target nucleic acids, two or more sensing parts may be located in a sensor for detecting target nucleic acids, and respective sensing parts may comprise liposomes comprising separate probes supported thereon for detecting individual target nucleic acids. For example, a first sensing part may comprise a liposome comprising a first probe and a liposome comprising a second probe designed for detecting a first target nucleic acid, and a second sensing part may comprise a liposome comprising a third probe and a liposome comprising a fourth probe designed for detecting a second target nucleic acid. When the plurality of sensing parts are included, the passage (003) may have branches (004) for connecting the inlet (002) to separate outlets where each sensing part is located, or as long as each individual reaction does not interfere with each other, may be connected so that the fluid containing the sample and/or the surfactant sequentially passes through the individual sensing parts from the inlet (002) and moves to the outlet.

In particular, the sensor for detecting a target nucleic acid of the present invention may further comprise a house-keeping gene sensing part for detecting a house-keeping gene as one of the sensing parts.

In an embodiment of the present invention, the sensing part may comprise a first sensing part for detecting a house-keeping gene and a second sensing part for detecting a target nucleic acid. The house-keeping gene of the first sensing part refers to a gene capable of being easily and routinely used to normalize a gene expression pattern such as glyceraldehyde-3-phosphate dehydrogenase (GAPDH), Cypl, albumin, actin, tubulin, cyclophiiin hypoxantine phosphoribosyltransferase (HRPT), L32, 28S, 185, and the like. By using the house-keeping gene, it is possible to correct the signal of the target gene to correct the difference in an amount of quantitative genes for each individual, and it is possible to quantify and confirm the expression change pattern of the nucleic acid to be detected.

In other words, the first probe and the second probe of the first sensing part may contain sequences for detecting the house-keeping gene.

The second sensing part may be used to detect a target nucleic acid. The first probe and the second probe of the second sensing part may contain sequences for detecting a target nucleic acid.

The sensor for detecting a target nucleic acid of the present invention may be in the form of a microfluidic chip.

The microfluidic chip has the ability to simultaneously perform various experimental conditions by flowing fluid through the microfluidic channel. Specifically, the microchannel may be formed using a substrate (or chip material) such as plastic, glass, silicon, or the like, then a fluid (for example, liquid sample) may be moved through the formed channel, and reaction and detection may be performed through a plurality of sensing parts, and the like, in the microfluidic chip.

In an embodiment of the present invention, disclosed is a sensor for detection in the form of a microfluidic chip, wherein a sensing part having a size of approximately 8 mm was formed through swelling of a 6 mm hydrogel, and a height thereof was set to approximately 1 mm. The entire chip had a structure set to approximately 65 mm in width and 25 mm in length.

Accordingly, the microfluidic chip preferably has a size of about 50 to 100 mm in a (horizontal) direction in which the fluid flows, and preferably has a size of about 15 to 40 mm in a vertical direction. The hydrogel preferably has a diameter size of approximately 4 mm to 12 mm, and a height of approximately 0.5 mm to 2 mm.

The detection composition, hydrogel, detection method, kit, and detection sensor of the present invention may be used for various detection purposes, and when the target nucleic acid is a biomarker for disease diagnosis, it is also possible to be usefully employed for rapid and accurate diagnosis of various diseases.

Hereinafter, the present disclosure will be described in more detail through Examples. However, these Examples are provided to illustrate the present disclosure by way of example, and the scope of the present disclosure is not limited to these Examples.

### MODE FOR CARRYING OUT THE INVENTION

### <Example 1> Preparation of Polyethylene Glycol Diacrylate (PEGDA)

60 g of Polyethylene glycol (PEG) was dissolved in 75 mL of dichloromethane (DCM). It was confirmed that the solution became transparent, and 7 mL of N,N-diisopropylethylamine (DIPEA) was then added to the solution. While maintaining the glass containing the solution at 4° C., 6.5 mL of acryloyl chloride was added. This reaction was refluxed under nitrogen and performed in a shaded place for 8 to 12 hours. 1 L of Diethyl ether was added to the reaction mixture to obtain a precipitate, which was dried in a vacuum chamber. The dried compound was additionally dissolved in 75 mL of dichloromethane and 500 mL of 2 mol potassium carbonate (K₂CO₃) and reacted for 8 to 12 hours, followed by precipitation by adding 1 L of diethyl ether to thereby obtain polyethylene glycol diacrylate. Then, the precipitate was dried in a vacuum chamber to obtain a resulting product in a powder form.

This product was prepared to be used as a hydrogel of PEGDA material.

### <Example 2> Design of Self-Signal Amplifying DNA Probe for mRNA Detection

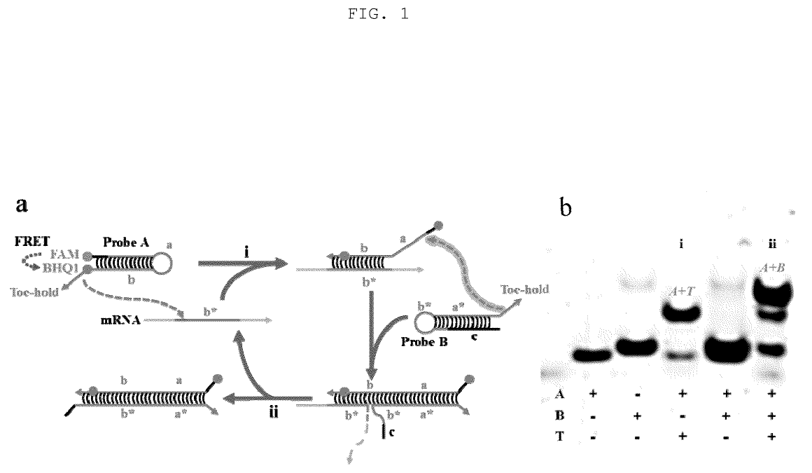

The reaction principle of catalytic hairpin assembly (CHA) according to the present invention is shown in FIG. 1a. FIG. 1a schematically shows the CHA circuit composed of probe A (PA) and probe B (PB) for signal amplification in a hydrogel. The PA strand of the circuit is modified at each end with a fluorophore (FAM) and a quencher (BHQ1), respectively, and the target mRNA initiates fluorescence recovery (i) and catalyzes the assembly of PA and PB via a toehold-mediated hairpin DNA circuit (ii).

Probes A and B having a hairpin structure were designed. The nucleotide sequences of the designed probes are shown in Table 1.

TABLE 1
All oligonucleotides

| | Sequences of oligonucleotide |
|---|---|
| name | (5′ to 3′) |
| A-ERBB2 | (FAM)AAA GCG ACC CAT TCA GGC ACC |
| (SEQ ID NO: 1) | GAG AAC AAA AGC TGA ATG GGT CGC |
| | TTT T(BHQ1)GT TCT |
| B-ERBB2 | CGA CTA CCT CAG GCA CCG AGA ACA |
| (SEQ ID NO: 2) | AAA GCG ACC CAT TCA GCT TTT GTT |
| | CTC GGT GCC TGA ATG GGT |
| A-GAPDH | (FAM)GTG AAG GTC GGA GTC AGT TAG |
| (SEQ ID NO: 3) | TGG GAA GGT GAT GAC TCC GAC CTT |
| | CAC CT(BHQ1)T CCC |
| B-GAPDH | AGG TAT GCG TCA GTT AGT GGG AAG |
| (SEQ ID NO: 4) | GTG AAG TCC GAC ACC TTC CCA CTA |
| | ACT GAC GTC GGA GTC ATC |
| *T-ERBB2 | TAA GAA CAA AAG CGA CCC ATT CAG |
| (SEQ ID NO: 5) | |
| **1MS-ERBB2 | TAA GAA CAA AA [symbol] CGA CCC ATT CAG |
| (SEQ ID NO: 6) | |
| ***2MS-ERBB2 | TAA GA [symbol] CAA AA [symbol] CGA CCC ATT CAG |
| (SEQ ID NO: 7) | |
| T-GAPDH | TGG GGA AGG TGA AGG TCG GAG TCA |
| (SEQ ID NO: 8) | |
| 1MS-GAPDH | TGG GGA AG [symbol] TGA AGG TCG GAG TCA |
| (SEQ ID NO: 9) | |
| 2MS-GAPDH | TG [symbol] GGA AG [symbol] TGA AGG TCG GAG TCA |
| (SEQ ID NO: 10) | |

*T: Target sequence,
**1MS: 1 base mismatched sequence,
***2MS: 2 base mismatched sequence,
italic: mismatched base

The probes were designed according to the theory of non-enzymatic fluorescence signal amplification. 6-Carboxylfluorescein (6-FAM) was bound to the 5′ end of the nucleotide sequence of probe A. A quencher blackhole quencher-1 (BHQ1) was bound to the 3′ end of the nucleotide sequence of probe A. The probes were boiled at 90° C. for 5 minutes and then slowly cooled at room temperature to annealing All probes were stored frozen until use.

Mutual binding of the designed probe groups was confirmed by performing polyacrylamide gel electrophoresis (PAGE). The electrophoresis gel was prepared with 10% acrylamide and run for 90 minutes under a voltage of 80 V using 1×TBE buffer. Then, the DNA was stained with GelRed® for 10 minutes to mark the location of the DNA, and then photographed with Gel-Doc (Bio-Rad Laboratories, Inc.) system.

The above reaction was confirmed by gel electrophoretic analysis using the synthesized probe set. Specifically, mutual binding of the designed probe groups was confirmed by performing polyacrylamide gel electrophoresis (PAGE). The electrophoresis gel was prepared with 10% acrylamide and run for 90 minutes under a voltage of 80 V using 1×TBE buffer. Then, the DNA was stained with GelRed® for 10 minutes to mark the location of the DNA, and then photographed with Gel-Doc (Bio-Rad Laboratories, Inc.) system.

Results thereof are shown in FIG. 1b.

As confirmed in FIG. 1b, it could be confirmed that the reaction proceeded sequentially by the above probe set at room temperature (+ indicates presence of, − indicates absence of).

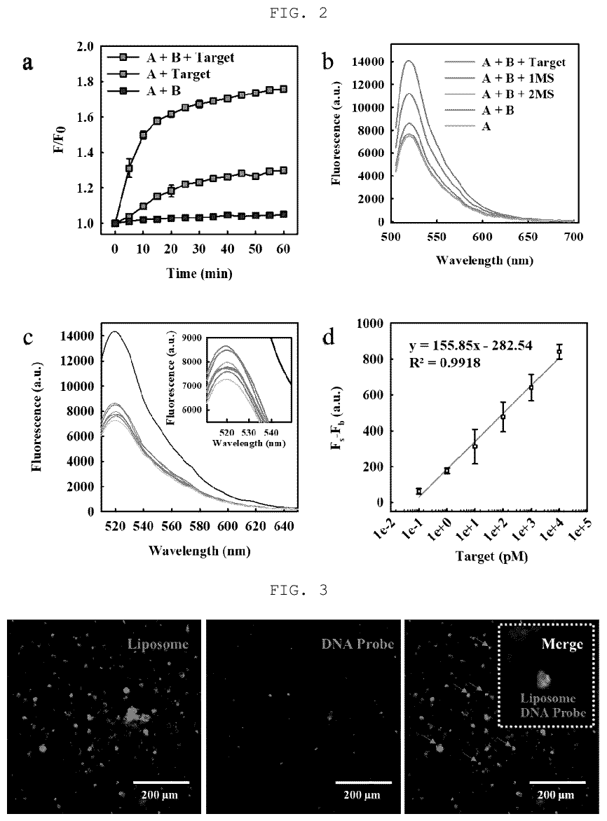

The form and result of this reaction were confirmed in more detail through fluorescence analysis, and results thereof are shown in FIG. 2.

According to FIG. 2a, it was shown that due to the role of probe B, a larger amount of fluorescence signal was generated within the same time (A+B+Target compared to A+Target), and that the fluorescence signal was stably maintained in the target-free condition (A+B).

In addition, as shown in FIG. 2b, as a result of confirming the selectivity of the detection probe using the Experimental Group DNA (Control) in which one (1MS) or 2 (2MS) nucleotide sequence was replaced from the target gene, the reaction with the target gene showed the highest fluorescence, which had a large difference from the fluorescence of the control gene reaction.

Further, as shown in FIG. 2c, the probe group was treated with a synthetic target at a concentration of 100 nM to 100 fM, and the fluorescence value was measured, and after reacting for 2 hours, the fluorescence was measured, wherein the detection limit could be confirmed to be 1.00 pM.

It was confirmed from these results that the catalytic hairpin assembly (CHA) system according to the present invention exhibits excellent effects in detecting target genes.

### <Example 3> Preparation of Probe-Supported Liposome

Liposomes were prepared by the conventional lipid film hydration method. To 10 mL of chloroform solution, 7 mg of phosphatidylcholine (PC), 0.7 mg of dioleoyl-3-trimethylammonium propane (DOTAP), and 1.95 mg of cholesterol (5-cholesten-3β-ol) were dissolved. A thin lipid film was prepared by evaporating the solvent using a rotary vacuum evaporator at room temperature. After adding 1 mL of 10 nM oligonucleotide solution in TE buffer to the lipid film, the lipid film was separated from the glass using a vortex mixer The solution was stored at 4° C. for 8 to 12 hours. In order to remove unencapsulated oligonucleotides, the solution was filtered through an Amicon centrifugal filter at 4° C. and 4000 rpm for 60 minutes.

In order to confirm that the prepared probe was encapsulated in the liposome, a DNA probe with green fluorescence (FAM) was encapsulated in the liposome and observed through a fluorescence microscope using a liposome dye (dye_red).

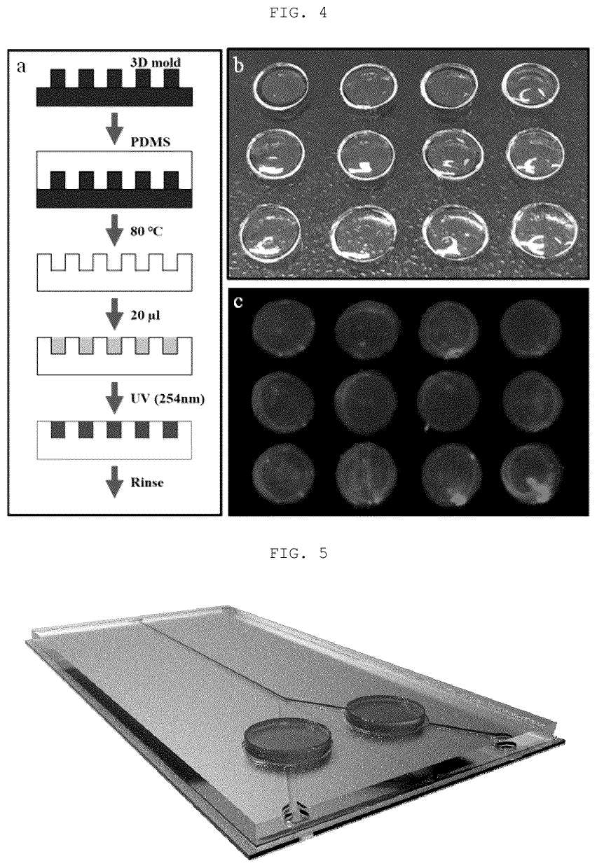

Results thereof are shown in FIG. 3.

As could be confirmed in FIG. 3, two types of fluorescence were seen at the same location, thereby confirming that the probe was encapsulated in the liposome.

### <Example 4> Production of Hydrogel Containing Probe-Supported Liposome

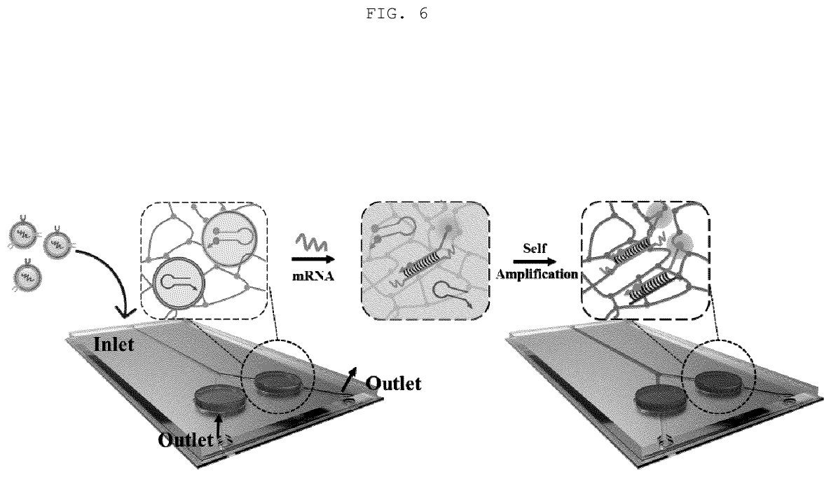

A cylindrical mold for forming the shape of the hydrogel was produced using 3D printing, and the specific mold formation process using polydimethylsiloxane (PDMS) is shown in FIG. 4a. PDMS was placed into a cylindrical mold and heated to 80° C. to cure the PDMS, and then separated to manufacture a mold. A hydrogel was produced using the manufactured mold in the following manner. Polyethylene glycol diacrylate at a weight ratio of 20%, polyethylene glycol at a weight ratio of 20%, liposomes encapsulated with 10 picomoles of the probes, and 2-hydroxy-2-methylpropiophenone (HMPP) at a weight ratio of 0.1% were combined. The prepared solution was exposed to an ultraviolet lamp (254 nm) for about 2 minutes to produce a hydrogel through photopolymerization. The produced hydrogel was soaked in sterile water (DW) for 2 hours to remove non-encapsulated liposomes.

The above overall process and confirmation results are shown in FIG. 4.

FIG. 4a shows the entire process of producing the PEGDA hydrogel, and FIGS. 4b and 4c show images of the produced hydrogel.

### <Example 5> Manufacturing of Microfluidic Chip

A casting mold (height: 100 μm) was manufactured by fabricating patterns using SU-8 photosensitive resin on a silicon wafer, as shown in FIG. 5. A structure having a diameter of 6 millimeters and a height of 1 millimeter produced by a 3D printer was attached to the manufactured casting mold, then liquid polydimethylsiloxane (PDMS) was solidified in the casting mold to form a chip, and the chip was attached to a slide glass, thereby manufacturing a microfluidic channel device.

The produced hydrogel was placed on the device and used for genetic analysis.

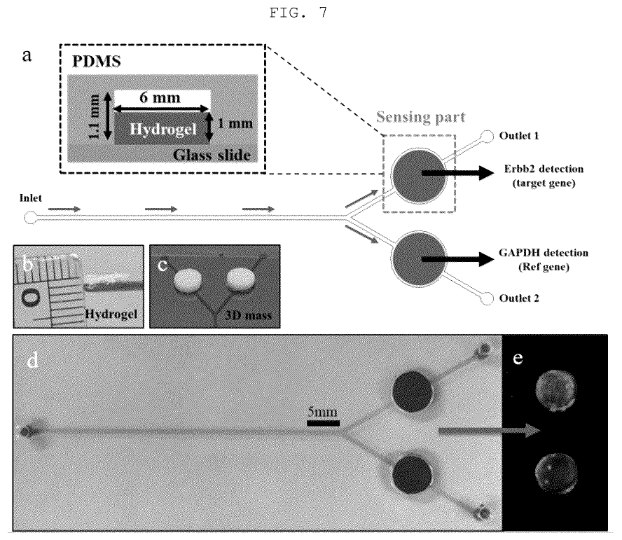

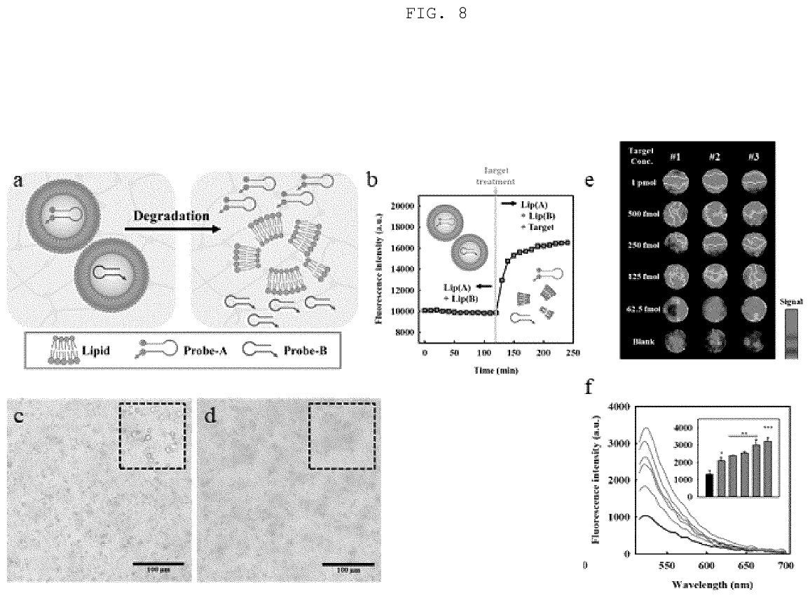

Specifically, the analysis principle using the microfluidic chip is shown in FIGS. 6 and 7.

As could be confirmed in FIG. 6, the sample and surfactant introduced through the inlet are directed to the sensing part of the outlet through the microtubule passage. When the surfactant reaches the sensing part together with the sample, the liposomes are degraded to release the probes, and the reaction between the probes and the detection gene results in a change in fluorescence development.

Further, in FIG. 7, the above description is shown in detail. FIG. 7a shows the size of an exemplary sensing part, and FIG. 7b to 7d show sizes and configurations thereof.

Specifically, a sensing part of approximately 8 mm was constructed through swelling of a 6 mm hydrogel, wherein a height thereof was set to about 1 mm.

The entire chip had a structure set to approximately 65 mm in width and 25 mm in length.

### <Example 6> Evaluation of Hydrogel Containing Probe-Supported Liposome

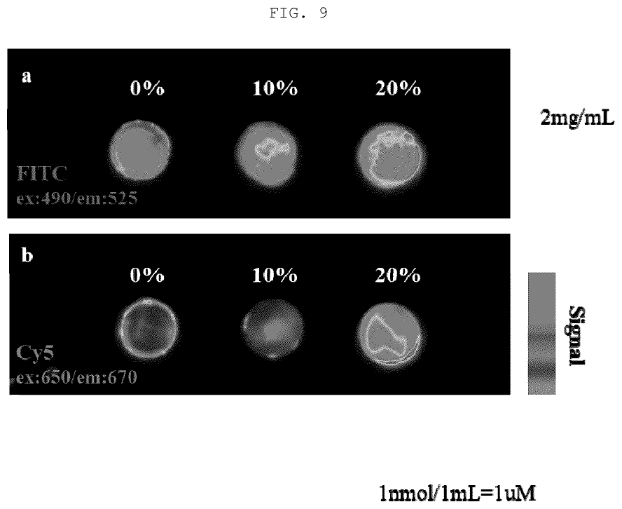

A schematic diagram of a hydrogel containing a probe-supported liposome according to the present invention is shown in FIG. 8A. As could be confirmed in FIG. 8a, the probes in the liposomes may be released into the hydrogel as the liposome membrane is separated by the surfactant treatment, and the reaction may be performed.

This expression change was confirmed through fluorescence change analysis.

Specifically, the liposomes were treated with 1% Triton X-100 at a certain time point, and the fluorescence intensities were measured by fixing the measurement wavelength of the fluorescence equipment (λ=484 nm) and putting the sample into a 96-well plate.

Results thereof are shown in FIG. 8b. As could be seen in the drawing, the treatment with 1% Triton X-100 degraded the liposomes and allowed two types of probes to be released, which resulted in a change in fluorescence response.

In addition, the breakdown of the liposomes was confirmed through a microscope.

Results thereof are shown in FIG. 8c (before surfactant treatment) and FIG. 8d (after surfactant treatment). As could be confirmed from the results, it could be confirmed that the liposome was degraded by the treatment with the surfactant.

In addition, the target gene synthesized in Example 2 was treated in a concentration-dependent manner at a very low treatment concentration of 62.5 fmol to 1 pmol.

Results thereof are shown in FIGS. 8e and 8f. As could be confirmed in the drawing, the synthesized target gene was treated and as a result, it could be confirmed that the fluorescence was changed in a concentration-dependent manner.

### <Example 7> Optimization Progress of Hydrogel

Polyethylene glycol (PEG) may be added together at the time of producing hydrogel and may play a role in creating pores inside. Therefore, since the number of pores increases as the concentration increases, it is necessary to find the most suitable conditions for detection by adjusting the amount of pores (pores) inside the hydrogel.

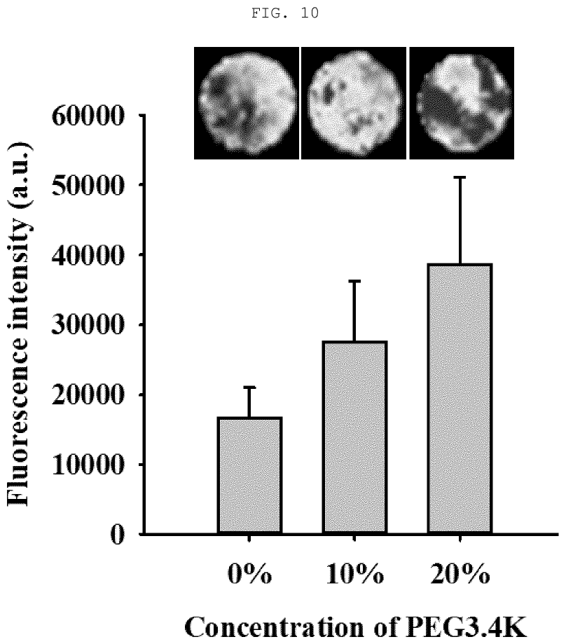

Accordingly, the fluorescence change was confirmed while changing the PEG conditions under the same conditions as in Example 4, and results thereof are shown in FIG. 9.

As could be seen in FIG. 9, it was confirmed that when mRNA diffusion was confirmed using 2 mg/mL FITC-Dextran70K (almost 12 nm of diameter) and 1 uM Cy5 modified oligonucleotides (20 nt), the most appropriate diffusion result was obtained at 20%.

In addition, the same amount of the target was treated with the hydrogel produced under each PEG condition, and reacted at the same time, and then the fluorescence values were measured.

Results thereof are shown in FIG. 10. As could be confirmed in FIG. 10, it could be confirmed that the reaction between the detection probes increased as the concentration of PEG increased.

In other words, it was confirmed from the above results that PEG was preferably used in an amount of about 20% by weight, and more preferably, PEG and PEGDA were used in a weight ratio of about 1:1.

### <Example 8> Confirmation of Expression Level in Cell Line

Two cell lines over-expressing HER2 (human epidermal growth factor receptor 2), a biomarker protein specific to breast cancer, i.e., HCC1954 (ATCC® CRL-2338¹) and SK-BR-3 (ATCC® HTB-30™), and three types of HER2 normal-expressing cell lines, i.e., HCC1143 (ATCC® CRL-2321™), MCF7 (ATCC® HTB-22™), and MDA-MB-231 (ATCC® HTB-26™) were used as Experimental Groups and Control groups. HCC1954, HCC1143, and SK-BR-3 cell lines were cultured in RPMI-1640 medium supplemented with 10% fetal bovine serum (FBS) and 1× penicillin-streptomycin. MCF7 and MDA-MB-231 cell lines were cultured in Dulbecco's modified eagle's medium (DMEM medium) supplemented with 10% fetal bovine serum (FBS) and 1× penicillin-streptomycin.

Exosomes were extracted from the cells and the expression level of the ERBB2 gene was confirmed.

In order to perform the isolation of exosomes, the cell lines were cultured in exosome-free culture medium for 48 hours and the medium was collected. Centrifugation was performed at 300 gravitational acceleration (xg) for 10 minutes, and a 0.45 micrometer filter was used to remove impurities. The blood of the disease model mouse was centrifuged at 1,500 gravitational acceleration for 15 minutes, and the supernatant, plasma, was collected. The plasma was centrifuged at 16,000 gravitational acceleration for 30 minutes and impurities were removed using a 0.45 micrometer filter. ½ Volume of Exo-spin™ buffer (Cell guidance systems) was added to the collected medium or plasma, and then refrigerated for 2 hours. The mixed solution was centrifuged for 1 hour at 16,000 gravitational acceleration. Then, the supernatant was removed and the precipitate was redispersed with TNaK buffer.

Then, quantitative reverse transcriptase PCR (qRT-PCR) was performed. Cellular RNA was extracted using the RNeasy Mini Kit (Qiagen). Exosomal RNA and plasma RNA were extracted using the ExoRNeasy Maxi kit (Qiagen) The concentration of extracted RNA was measured using a spectrophotometer (Nanodrop 2000, Thermo). The extracted RNA was reverse transcribed using the miScript II RT kit to synthesize cDNA, and PCR was performed according to the miScript SYBR Green PCR Kit (Qiagen). The mRNA analysis was performed on CFX96 Real-Time equipment (Bio-rad), and all experiments were repeated 3 times. Each sample was subjected to normalization with GAPDH (a house-keeping gene) to obtain quantitative results.

Thus, amounts of ERBB2 gene expression in HER2 over-expressing/normal cells and cell-derived exosomes were confirmed.

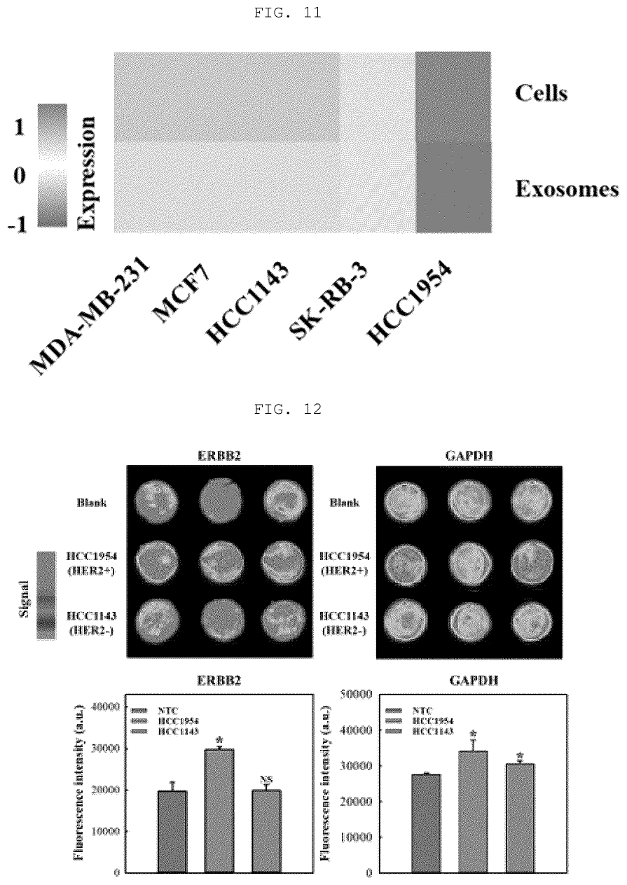

Results thereof are shown in FIG. 11.

As could be from in FIG. 11, HCC1954 showed a significant over-expression of the ERBB2 gene, and thus this cell line and the under-expressing cell line HCC1143 were used in subsequent experiments.

After extracting exosomes derived from HER2 over-expressing cells (HCC1954) and HER2 under-expressing (HCC1143) cells, experiments were performed on the hydrogel.

Specifically, the exosomes extracted from the cells were treated on the hydrogel produced in Example, and then a change in fluorescence expression was confirmed. In particular, the level of quantitative change in expression was also confirmed by using GAPDH together as the house-keeping gene, and results thereof are shown in FIG. 12.

As could be confirmed in FIG. 12, it was confirmed that ERBB2 was relatively highly increased in the exosomes of HER2-over-expressing cells, and GAPDH showed a similar pattern as the house-keeping gene.

### <Example 9> Confirmation of Detection Results in Animal Models

Human breast cancer cell line HCC1954 (6×10⁶) was injected into the breast tissue of BALB/c nude mice (female) by 29G insulin injection, and tumors were observed until they grew to a size of 1000 mm³ or died. The size of the implanted tumor was measured three times a week and was calculated as (4/3) XπX(short axis/2) 2 X(long axis/2) mm³. All animal experiments were conducted under the approval of the Institutional Animal Care and Use Committee (IACUC 2017-0329) of the Laboratory Animal Research Center of Yonsei University. 4-week-old female BALB/c nude mice (N=5, average body weight of 21±6 g) were purchased from Central Lab Animal Inc. Each mouse was housed in a standard cage cleaned under an automatic air control system with temperature (22±2° C.), humidity (approximately 60%) and lighting (light/dark cycles at 12 h intervals). Each mouse was provided with food (Pico Lab 5053, LabDiet, CA, USA) and sterile water throughout the experiment.

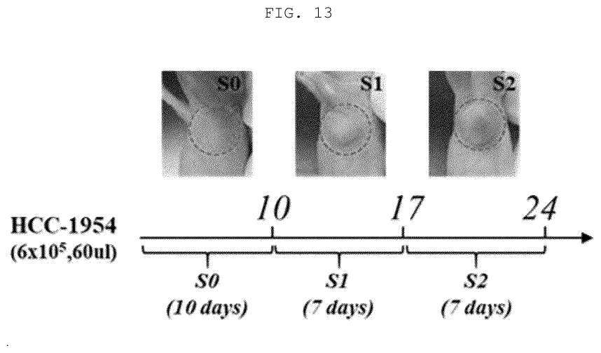

The change in tumor size by cycle during the schedule is shown in FIG. 13.

According to the above schedule, exosomes were extracted from rat urine in a similar manner as in Example 7 above, and the changes in expression were measured using qRT-PCR and the hydrogel according to the present invention.

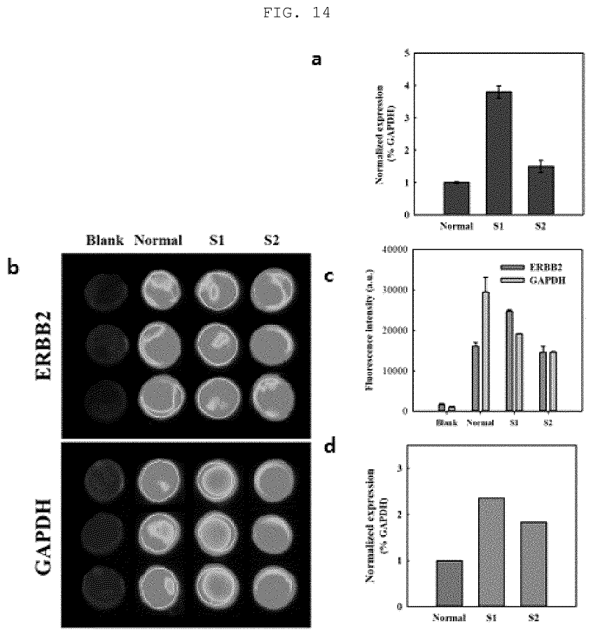

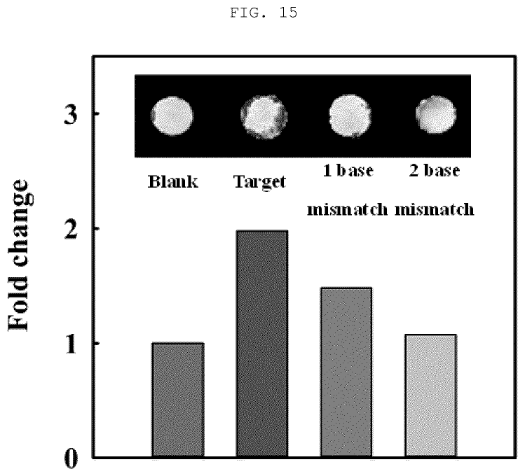

FIG. 14a shows PCR data of exosomes extracted from mouse urine (confirmation of quantitative expression of the ERBB2 gene). Similarly, whether or not the changes in expression were shown in the hydrogel was also confirmed through the results of using the hydrogel according to the present invention, and these results are shown in FIGS. 14b to 14d.

FIG. 14b shows the result of fluorescence measurement after treatment of the exosomes extracted from mouse urine with the hydrogel, and FIG. 14c shows the digitization results in a quantitative way. In addition, FIG. 14d shows the result of correcting the ERBB2 gene detection fluorescence values with the GAPDH fluorescence values. It could be confirmed from these results that compared to the control group (Normal), the fluorescence value significantly changed when the exosomes derived from the urine of disease model mice were treated, showing a similar tendency to the above PCR result of FIG. 14a, and thus the hydrogel had high applicability for diagnostic use.

### <Example 10> Evaluation of Accuracy of Hydrogel System Containing Probe-Supported Liposomes

The detection efficiency for the target sequence was compared with those of the mismatched target sequences prepared in Example 2 above. Specifically, similar to Example 6, the target gene and 1 and 2 mismatched sequences were treated on the hydrogel, respectively, and the change in detectability was confirmed.

Results thereof are shown in FIG. 15. As could be seen in FIG. 15, the target sequence showed excellent detection efficiency, but when mismatched sequences were contained, detection was not performed, and thus it could be confirmed that the hydrogel according to the present invention could be used to have remarkably good selectivity and accuracy.

### <Example 11> Application to Microfluidic System

Based on the microfluidic chip of Example 5, a change in gene expression in blood was confirmed using exosomes extracted from the mouse blood of Example 8 above.

Specifically, the inlet of the manufactured chip was blocked, and the inside of a vacuum chamber was evacuated by discharging the gas inside the chip for 30 minutes. After injecting about 100 μL of the sample solution through the inlet and reacting for 2 hours, fluorescence was measured with Gel-Doc system.

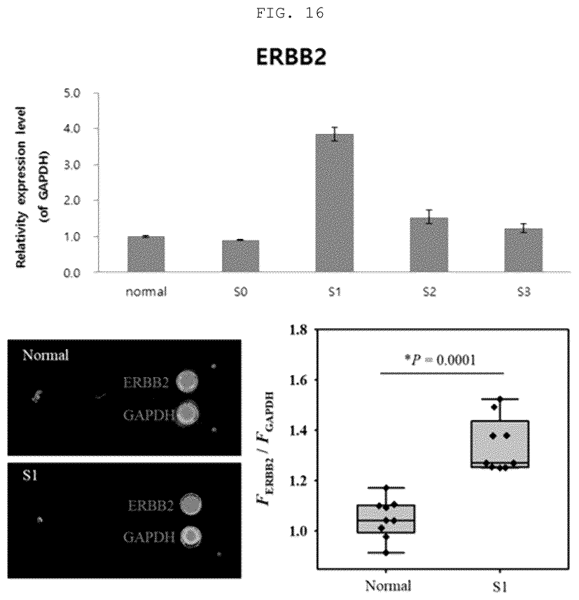

Results thereof are shown in FIG. 16, and FIG. 16 shows results of reflecting changes in ERBB2 expression over time in fluorescence and graphs.

Specifically, it was confirmed that the normal group and the HRBB2 over-expression group could be distinguished through gene expression changes corrected by GAPDH.

### <Example 12> Application to Microfluidic System

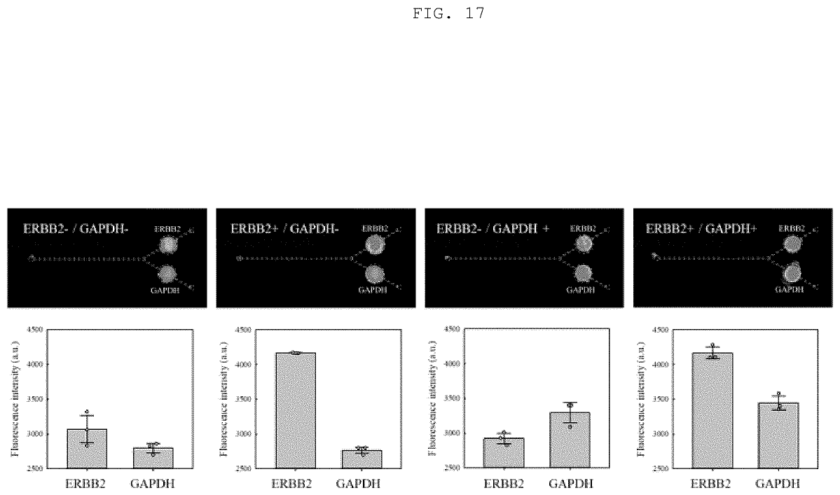

The microfluidic chip according to the present invention was used to confirm 1) target synthetic DNA of ERBB2 and GAPDH, 2) exosomes derived from HCC1141 and HCC1954, and 3) changes in exosome gene expression derived from mouse blood, and results thereof are shown in FIGS. 17 to 19. The target synthetic DNA is prepared by mimicking a portion of the RNA sequence of ERBB2 and GAPDH to be detected, and means that a target nucleic acid sequence capable of binding to a probe is prepared as DNA.

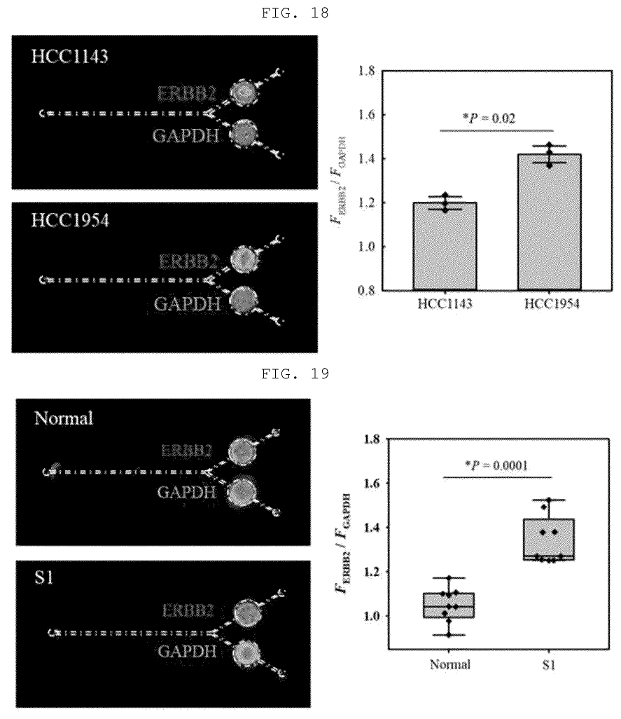

FIG. 17 shows results of confirming the change in fluorescence expression depending on the presence or absence of target synthetic DNA of ERBB2 and GAPDH, FIG. 18 shows changes in expression of exosomes derived from HCC1141 and HCC1954, and FIG. 19 shows results of treatment with blood-derived exosomes of the mouse animal model.

As could be confirmed in FIG. 17, the change in expression depending on the presence or absence of RNA of ERBB2 and GAPDH was clearly confirmed through the microfluidic system of the present invention, and it was confirmed that the correction of values could proceed together due to the presence of the house-keeping gene.

In addition, these results were also confirmed in cell line-derived exosomes as shown in FIG. 18 or animal blood-derived exosomes as shown in FIG. 19.

In other words, it could be confirmed that the microfluidic system according to the present invention was used to have an excellent effect in detection of trace amounts of genes.

From the above description, those skilled in the art to which the present disclosure pertains will understand that the present disclosure may be embodied in other specific forms without changing the technical spirit or essential characteristics thereof. In this regard, it should be understood that the embodiments described above are illustrative in all respects and not restrictive. As the scope of the present disclosure, it should be construed that all changes or modifications derived from the meaning and scope of the claims to be described below and equivalents thereof rather than the above detailed description are included in the scope of the present disclosure.

### DETAILED DESCRIPTION OF MAIN ELEMENTS

001 SENSOR FOR DETECTING TARGET NUCLEIC ACID
002 INLET
003 PASSAGE
004 BRANCH
005 SENSING PART

1-17. (canceled)

18. A composition for detecting a target nucleic acid comprising:
1) a first probe having a hairpin structure in which a reporter is conjugated to one end and a quencher is conjugated to the other end, and comprising an off-target sequence and a target sequence complementary to a target nucleic acid; and
2) a second probe comprising a sequence complementary to the first probe;
wherein the first probe and the second probe are each supported on separate liposomes.

19. The composition of claim 18, wherein the second probe has a hairpin structure.

20. The composition of claim 18, wherein the second probe comprises the target sequence and a sequence complementary to the off-target sequence of the first probe.

21. The composition of claim 18, wherein the reporter is any one selected from the group consisting of ALEX-350, FAM, VIC, TET, CAL Fluor® Gold 540, JOE, HEX, CAL Fluor Orange 560, TAMRA, CAL Fluor Red 590, ROX, CAL Fluor Red 610, TEXAS RED, CAL Fluor Red 635, Quasar 670, CY3, CY5, CY5.5, and Quasar 705, and
the quencher is any one selected from the group consisting of DABCYL, BHQ, ECLIPSE, and TAMRA.

22. The composition of claim 18, wherein the composition is a hydrogel composition.

23. A hydrogel for detecting a target nucleic acid comprising:
1) a first probe having a hairpin structure in which a reporter is conjugated to one end and a quencher is conjugated to the other end, and comprising an off-target sequence and a target sequence complementary to the target nucleic acid; and
2) a second probe comprising a sequence complementary to the first probe;
wherein the first probe and the second probe are each supported on separate liposomes.

24. The hydrogel of claim 23, wherein the hydrogel comprises one or more particles selected from the group consisting of natural polymers, acryl-based monomers or polymers, polyacrylamide-based monomers or polymers, phosphatidylcholine, hyaluronic acid-based monomers or polymers, carboxymethyl cellulose, alginate, chitosan, poly(e-caprolactone), poly(lactic acid), poly(glycolic acid), polyethylene glycol, hydroxyapatite, tricalcium phosphate, and mixtures thereof.

25. The hydrogel of claim 23, wherein the hydrogel comprises a mixture of polyethylene glycol and polyethylene glycol diacrylate.

26. The hydrogel of claim 23, wherein the hydrogel is photocured.

27. The hydrogel of claim 23, wherein the second probe has a hairpin structure.

28. The hydrogel of claim 23, wherein the second probe comprises the target sequence and a sequence complementary to the off-target sequence of the first probe.

29. The hydrogel of claim 23, wherein the reporter is any one selected from the group consisting of ALEX-350, FAM, VIC, TET, CAL Fluor® Gold 540, JOE, HEX, CAL Fluor Orange 560, TAMRA, CAL Fluor Red 590, ROX, CAL Fluor Red 610, TEXAS RED, CAL Fluor Red 635, Quasar 670, CY3, CY5, CY5.5, and Quasar 705, and
the quencher is any one selected from the group consisting of DABCYL, BHQ, ECLIPSE, and TAMRA.

30. A method for detecting/diagnosing a target nucleic acid, comprising:
reacting a sample and a surfactant with the composition according to claim 18.

31. The method of claim 30, wherein the liposome containing the first probe and the liposome containing the second probe are simultaneously contained in a hydrogel.

32. The method of claim 30, wherein the second probe has a hairpin structure.

33. The method of claim 30, wherein the second probe comprises the target sequence and a sequence complementary to the off-target sequence of the first probe.

34. A sensor for detecting a target nucleic acid comprising
an inlet part;
at least one sensing part containing a composition for detecting a target nucleic acid; and
a passage part connecting the inlet part and the sensing part,
wherein the composition for detecting a target nucleic acid comprising:
1) a first probe having a hairpin structure in which a reporter is conjugated to one end and a quencher is conjugated to the other end, and comprising an off-target sequence and a target sequence complementary to a target nucleic acid; and
2) a second probe comprising a sequence complementary to the first probe;
wherein the first probe and the second probe are each supported on separate liposomes.

35. The sensor of claim 34, wherein the composition is included in the form of a hydrogel in the sensing part.

36. The sensor of claim 34, further comprising:
a house-keeping gene sensing part for detecting a house-keeping gene.

37. The sensor of claim 34, wherein the sensor is a microfluidic chip.


SEQUENCE LISTING (10 sequences)

SEQ ID NO: 1 - DNA, 54 bp, Artificial Sequence
  A-ERBB2
aaagcgacccattcaggcaccgagaacaaaagctgaatgggtcgcttttgttct

SEQ ID NO: 2 - DNA, 66 bp, Artificial Sequence
  B-ERBB2
cgactacctcaggcaccgagaacaaaagcgacccattcagcttttgttctcggtgcctgaatgggt

SEQ ID NO: 3 - DNA, 54 bp, Artificial Sequence
  A-GAPDH
gtgaaggtcggagtcagttagtgggaaggtgatgactccgaccttcaccttccc

SEQ ID NO: 4 - DNA, 66 bp, Artificial Sequence
  B-GAPDH
aggtatgcgtcagttagtgggaaggtgaaggtcggagtcatcaccttcccactaactgactccgac

SEQ ID NO: 5 - DNA, 24 bp, Artificial Sequence
  T-ERBB2
taagaacaaaagcgacccattcag

SEQ ID NO: 6 - DNA, 24 bp, Artificial Sequence
  1MS-ERBB2
taagaacaaaaccgacccattcag

SEQ ID NO: 7 - DNA, 24 bp, Artificial Sequence
  2MS-ERBB2
taagatcaaaatcgacccattcag

SEQ ID NO: 8 - DNA, 24 bp, Artificial Sequence
  T-GAPDH
tggggaaggtgaaggtcggagtca

SEQ ID NO: 9 - DNA, 24 bp, Artificial Sequence
  1MS-GAPDH
tggggaagctgaaggtcggagtca

SEQ ID NO: 10 - DNA, 24 bp, Artificial Sequence
  2MS-GAPDH
tgcggaagctgaaggtcggagtca